(19) United States
(12) Patent Application Publication
(10) Pub. No.: US 2019/0241606 A1
(43) Pub. Date: Aug. 8, 2019

(54) Process for preparation of cangrelor tetrasodium and intermediate therefor

(71) Applicant: ScinoPharm Taiwan, Ltd., Tainan (TW)

(72) Inventors: Tsung-Yu Hsiao, Tainan (TW); Chen-Wei Lin, Tainan (TW); Yu-Hui Huang, Tainan (TW); Meng-Fen Ho, Tainan (TW); Kuan-Hsun Wang, Tainan (TW)

(73) Assignee: ScinoPharm Taiwan, Ltd.

(21) Appl. No.: 15/888,182

(22) Filed: Feb. 5, 2018

(51) Int. Cl. C07H 19/20

(52) U.S. Cl. CPC C07H 19/20

(57) ABSTRACT

A process for preparing cangrelor tetrasodium comprising:
a) reacting a compound of formula M1 with morpholine to form a compound of formula M2; and

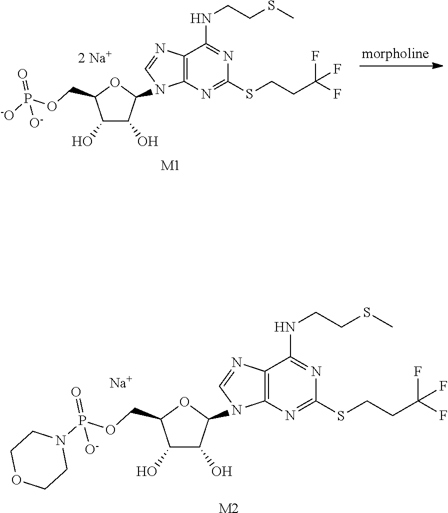

b) reacting the compound of formula M2 with clodronic acid to provide cangrelor tetrasodium

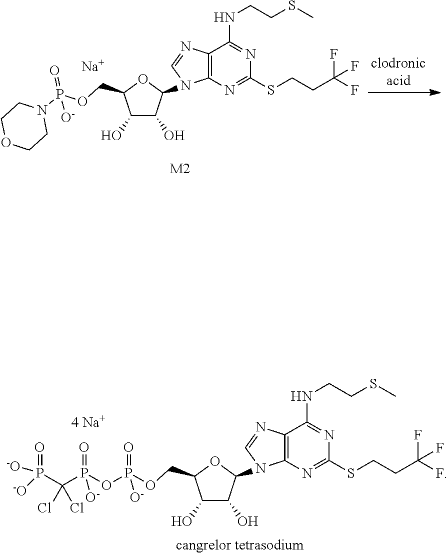

### BACKGROUND OF THE INVENTION

### 1. Field of the Invention

The present application relates to a process for preparation of cangrelor tetrasodium and intermediate therefor.

### 2. Description of the Related Art

Cangrelor tetrasodium is tetrasodium salt of N6-[2-(methylthio)ethyl]-2-[(3,3,3,trifluoropropyl)-5′-adenylic acid, monanhydride with (dichloromethylene) bisphosphonic acid, with an empirical formula of C₁₇H₂₁N₅C₁₂F₃Na₄O₁₂P₃S₂, a molecular weight of 864.3 g/mol, and a chemical structure represented below:

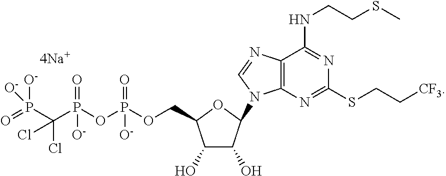 [SMILES: CSCCNc1nc(SCCC(F)(F)F)nc2c1ncn2[C@@H]1O[C@H](COP(=O)([O-])OP(=O)([O-])C(Cl)(Cl)P(=O)([O-])[O-])[C@@H](O)[C@H]1O]

Cangrelor tetrasodium (formerly known as AR-C69931MX) is a potent, selective, reversible inhibitor of ADP-induced platelet aggregation (P₂Y₁₂ inhibitor). It is indicated as an adjunct to percutaneous coronary intervention (PCI) for reducing the risk of periprocedural myocardial infarction (MI), repeat coronary revascularization, and stent thrombosis (ST) in patients who have not been treated with a P2Y12 platelet inhibitor and are not being given a glycoprotein IIb/IIIa inhibitor (1). Cangrelor tetrasodium was developed and marketed by The Medicines Company as Kengrexal® (in EU) or Kengreal® (in the US). Kengreal® is available as a lyophilized powder for reconstitution for injection, containing Eq. 50 mg of free cangrelor. Cangrelor has a unique efficacy and safety profile in comparison with currently available ADP (adenosine diphosphate) antagonists, such as clopidogrel, as well as GP IIb/IIIa antagonists. Cangrelor's short plasma half-life yields a rapid loss of activity following discontinuation of the infusion, which is a potentially significant safety advantage. It is designed to prevent platelet activation and aggregation that leads to thrombosis in acute care settings, including in patients undergoing percutaneous coronary intervention. Cangrelor tetrasodium is an adenosine triphosphate (ATP) analog modifying the side chain on the structure of purine and triphosphate.

U.S. Pat. No. 5,721,219 and Ingall et al., J. Med. Chem. 1999, 42, 213-220 (hereinafter “Ingall”) both disclose the preparation of cangrelor triammonium and its analogues with different salt forms. U.S. Pat. No. 5,721,219 discloses preparation of cangrelor tetrasodium analogue by a synthetic route involving one-pot reaction (see Example 2 in U.S. Pat. No. 5,721,219) and Scheme 1 below:

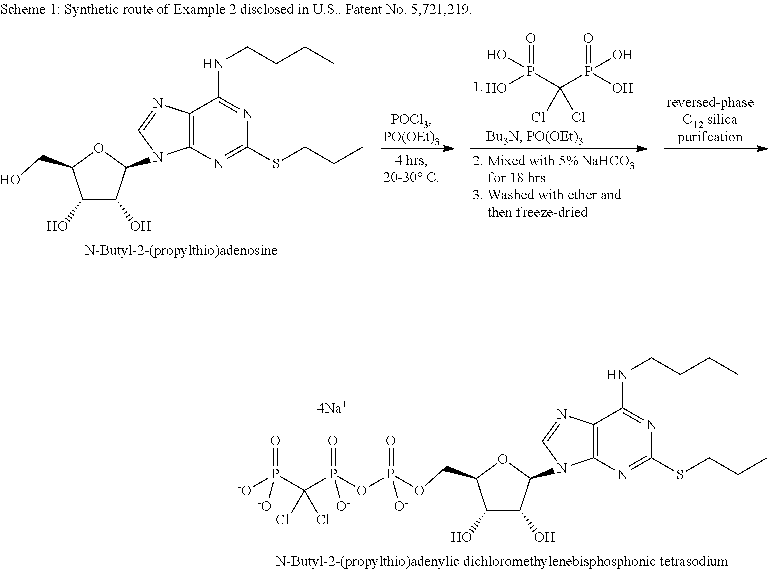

This one-pot reaction process does not isolate the phosphorylation product. After quenching and freeze-drying, the crude product is purified by reversed-phase chromatography to produce cangrelor tetrasodium analogue (N-butyl-2-(propylthio)adenylic dichloromethylenebisphosphonic tetrasodium).

U.S. Pat. No. 5,721,219 discloses preparation of cangrelor tetrasodium analogue by a synthetic route involving stepwise reaction (see Example 1 in U.S. Pat. No. 5,721,219 and Scheme 2 below:

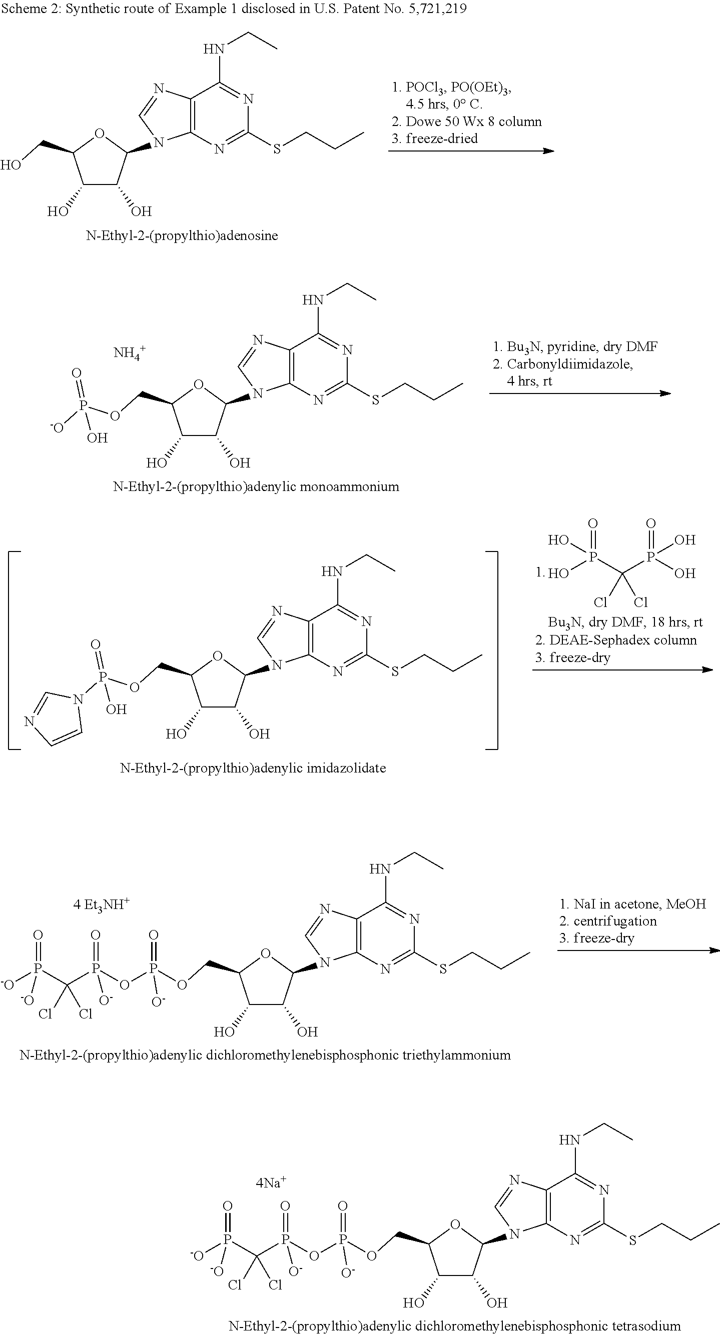

The first phosphorylation product resulting from the reaction between (N-ethyl-2-(propylthio)adenosine) and POCl₃/PO(OEt)₃ is isolated via ion-exchange chromatography (Dowex 50W×8, H+ form) and freeze-drying, then activated by carbonyldiimidazole and converted to N-ethyl-2-(propylthio)adenylic imidazolidate. This resultant unstable intermediate without isolation is coupled with dichloromethylenebisphosphonic acids. After continuously ion-exchange (DEAE-Sephadex) chromatography purification and freeze-drying, the desired N-ethyl-2-(propylthio)adenylic dichloromethylenebisphosphonic triethylammonium salt is provided. After salt transformation by Nal/acetone/MeOH, centrifugation and freeze-drying again, the desired product, N-ethyl-2-(propylthio)adenylic dichloromethylenebisphosphonic tetrasodium, is provided.

Although U.S. Pat. No. 5,721,219 does not disclose the yield of cangrelor triammonium (see Example 6b), it discloses a yield of 19.21% of an analog (see Example 2) through one-pot reaction approach and a yield of 25.34% of analog (see Example 1) through stepwise reaction approach. Moreover, Ingall discloses a yield of 4% of cangrelor triammonium (see Example 10l) through a synthetic route involving the same stepwise reaction disclosed in U.S. Pat. No. 5,721,219.

Therefore, there is a need for a convenient, low cost, and simple process of making cangrelor tetrasodium with a high yield.

### SUMMARY OF THE INVENTION

The present invention relates to a method for preparing cangrelor tetrasodium and intermediates therefor.

The first aspect of the present invention is a process for preparing cangrelor tetrasodium comprising:
a) reacting a compound of formula M1 with morpholine to form a compound of formula M2; and

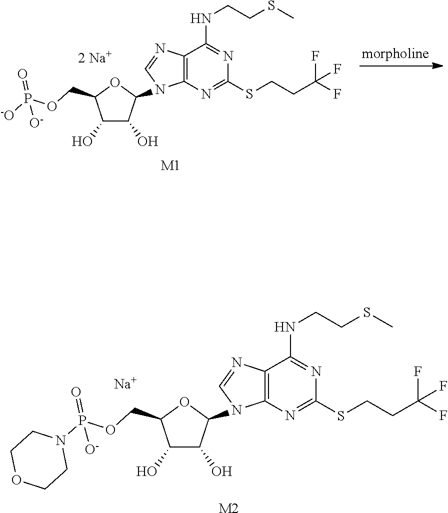

b) reacting the compound of formula M2 with clodronic acid to provide cangrelor tetrasodium

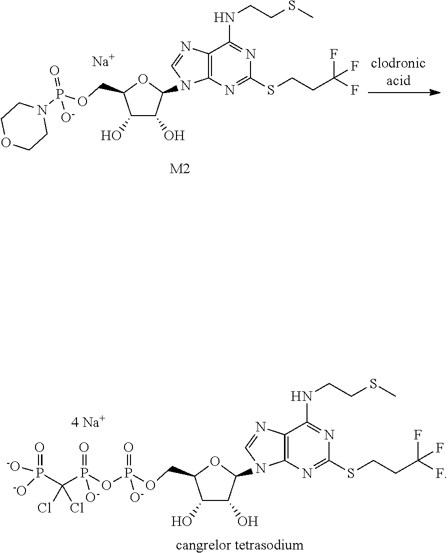

In this process, steps a) and b) may be conducted under any appropriate conditions. For example, step a) may be conducted at a temperature from 65 to 80° C., preferably 80° C., for 3 to 24 hours, preferably 3 to 4 hours.

The process may further comprise a step of phosphorylating a compound of formula SM1 to form the compound of formula M1:

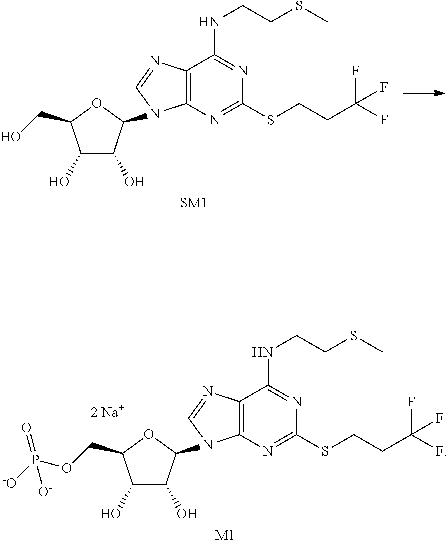

This phosphorylating step may be conducted under any appropriate conditions, for example, at a temperature from −10 to 25° C., preferably −10 to 0° C. for 3 to 24 hours, preferably 14 to 21 hours.

The compound of formula M1 may be isolated by precipitation in a solvent selected from the group consisting of DCM, acetone, THF, MTBE, IPE, EtOAc, IPAc, MeCN, MeOH, EtOH, IPA, t-BuOH, toluene, heptane, cyclohexane, water, and combinations thereof, preferably one of the following combinations:
a) MeOH and DCM;
b) MeOH and MTBE;
c) MeOH and IPE;
d) MeCN and water;
e) MeCN, water, and acetone; and
f) MeCN, water, and t-BuOH, and more preferably the combination of MeCN and water.

The compound of formula M2 in step a) may be modified by a workup technique selected from a group consisting of:
a) DCU filtration;
b) salt-exchanging with NaOH;
c) extraction with MeOH/MTBE or MTBE;
d) phosphate salt elimination; and combinations thereof.

The phosphate salt elimination may be conducted in acetone, MeCN, IPA, IPA together with MTBE, or IPA together with water, more preferably in IPA together with water.

The process may further comprise purifying the cangrelor tetrasodium obtained in step b) by a polystyrene/divinylbenzene resin, which, for example, may be one or more of HP20, HP20SS, SP20SS, HP21, SP825, SP850, and SP70, more preferably HP20SS. The polystyrene/divinylbenzene resin is eluted by a solution selected from the group consisting of H₂O, MeOH, EtOH, IPA, acetone, MeCN, MeOH/H₂O, EtOH/H₂O, MeCN/H₂O, 0-1% NH₄OH, 0-5% NH₄OAc, 0-1% NaCl, 0-1% NaOAc, 0-3% DMS, and combinations thereof. More preferably, the eluent is MeOH together with water or MeCN together with water.

The cangrelor tetrasodium formed in in step b) may be isolated by precipitation in a solvent selected from the group consisting of EtOH, IPA, DMSO, acetone, MeOH, MeCN, n-PrOH, t-BuOH, MTBE, and combinations thereof, preferably the solvent is selected from the following combinations:
a) MeCN/MeOH;
b) MeCN/EtOH;
c) MeCN/IPA;
d) acetone/MeOH;
e) acetone/EtOH;
f) acetone/DMSO;
g) IPA/MeOH;
h) n-PrOH/MeOH;
i) t-BuOH/MeOH; and
j) MeOH/MTBE. More preferably the solvent is MeCN or MeOH.

The reacting step b) may comprise: b1) reacting the compound of formula M2 with clodronic acid; b2) quenching the reaction mixture obtained in step b1) with ammonium hydroxide (NH₄OH) to obtain cangrelor tetraammonium; and b3) reacting the cangrelor tetraammonium with NaOH to provide cangrelor tetrasodium by salt-exchange:

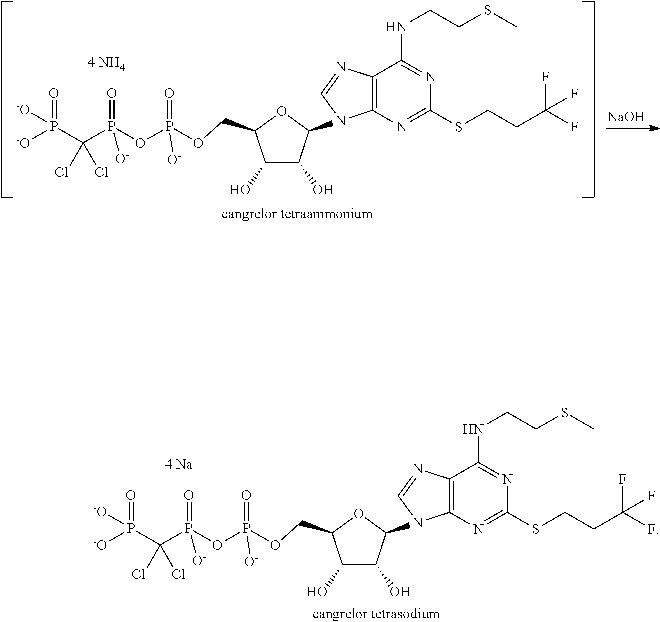

The second aspect of the present application is a compound of formula M2:

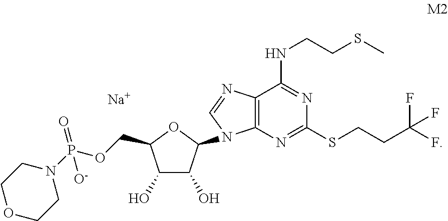 [SMILES: CSCCNc1nc(SCCC(F)(F)F)nc2c1ncn2[C@@H]1O[C@H](COP(=O)([O-])N2CCOCC2)[C@@H](O)[C@H]1O ; [Na+]]

As stated above, the compound of formula M2 may be used as an intermediate in making cangrelor tetrasodium.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### DETAILED DESCRIPTION OF THE PRESENTLY PREFERRED EMBODIMENTS

The following embodiments are provided to illustrate, but not to limit the instant invention.

The present invention relates to the development of a process for preparing cangrelor tetrasodium, which involves a stepwise reaction approach and improves the yield for over 10 times higher than the process disclosed in U.S. Pat. No. 5,721,219 and Ingall discussed above.

According to an embodiment of the instant invention and as shown in Scheme 3 below, the compound of formula SM1 is used as a starting material to react with phosphory chloride, and then the reaction mixture is treated with MeCN/H₂O, adjusted pH value with NaOH, precipitated M1 solid in MeCN/H₂O, and filtrated without further column purification. Subsequently, M1 is coupled with morpholine in the presence of dicyclohexylcarbodiimide (DCC) to generate M2 in t-BuOH/water. The subsequent workup procedure comprises dicyclohexyurea (DCU) filtration, salt-exchanging with NaOH, methyl tert-butyl ether (MTBE) extraction, phosphate salt elimination from isopropyl alcohol (IPA), and lyophilization without further column purification. The resulting M2 is coupled with clodronic acid in the presence of TEA and 4,5-dicyanoimidazole (DCI), and the resulting mixture is quenched with ammonia hydroxide, purified by HP20SS column, and concentrated by another HP20SS column to obtain a concentrated solution containing purified cangrelor tetraammonium. The HP20SS is porous polystyrene polymer resin with a large surface area to adsorb organic compounds. The process of loading crude cangrelor solution into HP20SS column and adsorbed by the HP20SS is “catch” stage. The process of desorption of cangrelor by eluent is “release” stage. The composition of eluent affects the effusion timing of cangrelor and the concentration of effluent. The HP20SS column could achieve different purposes by controlling the composition of eluent. The resulting solution containing pure cangrelor tetraammonium is salt-exchanged with NaOH and precipitated in MeOH to give a pure cangrelor tetrasodium solid without further lyophilization.

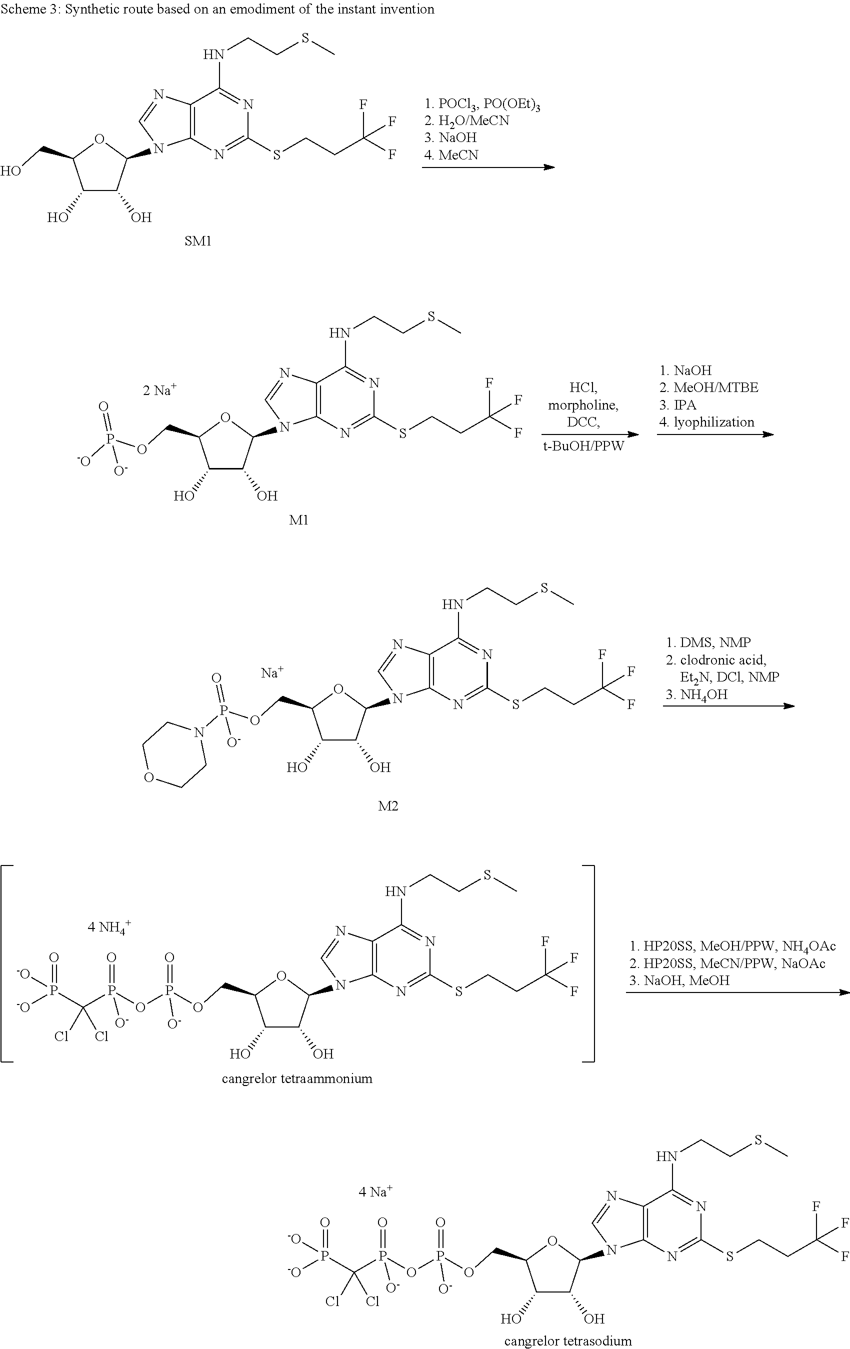

The technical features based on the embodiments of the instant invention are summarized as follows.

1. Based on an embodiment of the instant invention, M1 in the first step (i.e., phosphorylation) is isolated from precipitation in MeCN/H2O and filtration. U.S. Pat. No. 5,721,219 and Ingall isolate the compound through ion-exchange column (Dowex 50W) and lyophilization of the fractions.

2. According to an embodiment of the instant invention, the cangrelor tetrasodium is provided by involving the conversion from M1 to M2 and modified workup procedure (DCU filtration/salt exchange/MTBE extraction/phosphate salt elimination).

3. According to an embodiment of the instant invention, the cangrelor tetrasodium is purified by HP20SS column, and the collected fractions is also concentrated by the HP20SS column. U.S. Pat. No. 5,721,219 and Ingall disclose purification of the cangrelor tetrasodium via DEAE-Sephadex or reversed-phase (C12) column. The DEAE-Sephdex or reversed-phase (C12) column is not capable of concentrating the effluent and producing the diluted solution, and requires lyophilization to remove the solvent.

4. According to an embodiment of the instant invention, the cangrelor tetrasodium is salt-exchanged (NH₄⁺→Na⁺) in a homogenous solution (NaOH) and precipitated in MeOH. US. Pat. No. 5,721,219 and Ingall disclose salt-exchange by Nal/acetone under heterogenious condition and require further centrifugation and lyophilization to produce the final product.

The following Table A summarizes the advantages or characteristics of the embodiments of the instant invention compared with the processes reported in the art.

TABLE A
Comparison Table

| | | | | Advantage of the |
|---|---|---|---|---|
| | Relevant information to | Embodiments of | The | embodiments of this |
| Reference | this invention | this Invention | Difference | invention |
| U.S. Pat. No. 5,721,219 | One-pot reaction | Stepwise | Different | Having more process |
| | (example 6b) | reaction | synthetic | controls to ensure the |
| | | | route | quality |
| | Purification by a reversed | Purification by a | Different | 1. HP20SS is |
| | phase (C12) column, | HP20SS column | column and | cheaper than |
| | eluted by 4% salinein | eluted by 5-85% | eluents | reversed phase |
| | water and further by | MeOH/water | | C12 silica gel and |
| | DEAE-Sephadex column | (containing | | DEAE-sephadex |
| | eluted by 0-0.6M | 0.28% NH₄OH | | resin |
| | NH₄HCO₃ solution | and 1% | | 2. HP20SS is |
| | | NH₄OAc) | | capable of |
| | | | | accomplishing |
| | | | | catch-release |
| | | | | process to |
| | | | | concentrate |
| | | | | solution |
| | Final isolation from | Isolation from | Different | 1. Easier operation |
| | lyophilization | precipitation in | Isolation | without |
| | | MeOH and | method | lyophilization |
| | | filtration | | 2. Save time and |
| | | | | cost |
| Ingall | Stepwise reaction (the | Stepwise | Different | Yield is over 10 times |
| | preparation of Compound | reaction (42%) | synthetic | higher |
| | 10I, 4%) | | route | |
| | M1 intermediate is | M1 intermediate | Different | 1. Without involving |
| | lyophilized from the | is precipitated | isolation | column operation |
| | fractions of Dowex | and isolated | method | and lyophilization |
| | 50W × 8 column | from | | in purification |
| | | MeCN/water | | 2. Save time and |
| | | | | cost |
| | The imidazolide | The | Different | 1. The morpholidate |
| | intermediate is provided | morpholidate | reagents and | intermediate is |
| | by coupling M1 with CDI | intermediate is | solvents are | more stable than |
| | in anhydride DMF | provided by | used for | imidazolide (which |
| | | coupling M1 with | preparing the | cannot be |
| | | DCC/morpholine | intermediate | isolated) |
| | | in t-BuOH/water | | 2. The reaction is not |
| | | | | moisture sensitive |
| | Purification of cangrelor | Purification of | Column and | HP20SS is cheaper |
| | ammonium salt by | cangrelor | eluents are | than DEAE-sephadex |
| | DEAE-Sephadex column | ammonium salt | different | resin and is capable |
| | and eluted by 0-0.6M | by HP20SS | | of accomplishing the |
| | NH₄HCO₃ then | column eluted | | catch-release method |
| | lyophilization | by 5-85% | | to concentrate |
| | | MeOH/water | | solution |
| | | (containing | | |
| | | 0.28% NH₄OH | | |
| | | and 1% | | |
| | | NH₄OAc] | | |
| | Salt exchange (NH₄⁺ → | Salt exchange is | Different salt | 1. Salt exchange |
| | Na⁺) is carried out by | carried out by | exchange | step is |
| | using 1M Nal/acetone | using NaOH and | and isolation | homogenous |
| | and centrifugation, and | isolating by | method | 2. Easier operation |
| | isolating by lyophilization | precipitation in | | without involving |
| | | MeOH | | lyophilizaiton in |
| | | | | purification |

The following examples are provided to further illustrate, but not to limit, certain aspects of the present invention.

Abbreviations utilized in the present application are explained in the following table.

TABLE B
Abbreviation Table

| | |
|---|---|
| BSA | Benzenesulfonic acid |
| CDI | Carbonyldiimidazole |
| DCC | Dicyclohexylcarbodiimide |
| DCI | 4,5-Dicyanoimidazole |
| DCM | Dichloromethane |
| DCU | dicyclohexylurea |
| DIPEA | Diisopropyl ethyl amine |
| DMF | N,N-dimethylformamide |
| DMS | Dimethyl sulfide |
| DMSO | Dimethyl sulfoxide |
| EtOAc | Ethyl acetate |
| HCl | Hydrogen chloride |
| IPA | Isopropyl alcohol |
| IPE | Isopropyl ether |
| MeCN | Acetonitrile |
| MeOH | Methanol |
| MSA | Methanesulfonic acid |
| MTBE | Methyl tert-butyl ether |
| NaOH | Sodium hydroxide |
| NaHCO₃ | Sodium bicarbonate |
| NMI | N-methylimidazole |
| NMP | N-Methyl-2-pyrrolidone |
| POCl₃ | Phosphorus oxychloride |
| PO(OEt)₃ | Triethylphosphate |
| PPh₃ | Triphenylphosphine |
| PPTS | Pyrdinium p-toluenesulfonate |
| PPW | Process purified water |
| SPW | Soften potable water |
| TBA | Tributylamine |
| TEA | Triethylamine |
| TFA | Trifloroacetic acid |
| THF | Tetrahydrofuran |

### EXAMPLE 1

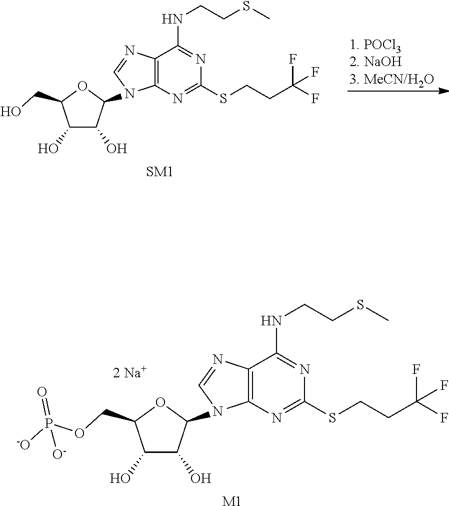

A compound of formula SM1 (200 g) and triethyl phosphate (PO(OEt)₃, 1.0 L) were added to a suitable vessel under nitrogen at 20-30° C. and stirred for 1 hour. The reaction mixture was cooled to—−10 to 0° C. Phosphorus oxychloride (POCl₃, 79.4 mL) was slowly added to the mixture at NMT0° C. for 1 hour. Then the reaction mixture was stirred at—−10-0° C. for 14 hours. After reaction was completed, the mixture was slowly added to pre-cooled co-solvent systems, water (6.0 L) and MeCN (2.0 L) at NMT0° C. for 30 minutes. After the addition was completed, the mixture was stirred at −10˜0° C. for 1 hour. An aqueous solution of 3 N NaOH (1.28 L) was slowly added at NMT0° C. to adjust the pH value till 8.0-8.5. After the adjustment was completed, the mixture was warmed to 10-15° C. Pre-cooled MeCN (32 L) was added to the mixture while maintaining internal temperature at NLT8° C. After the addition was completed, the mixture was stirred at this temperature for 1 hour. Then the mixture was cooled to 0-5° C. and stirred for 8 hours. The mixture was filtered and the filtered cake was washed with pre-cooled MeCN (2 L). The wet cake was suction dried with nitrogen purge for 10 hours and then dried under vacuum for 24 hours. A compound of M1 (238 g) was provided in 84% yield as pale skinny to white solid.

### EXAMPLE 2

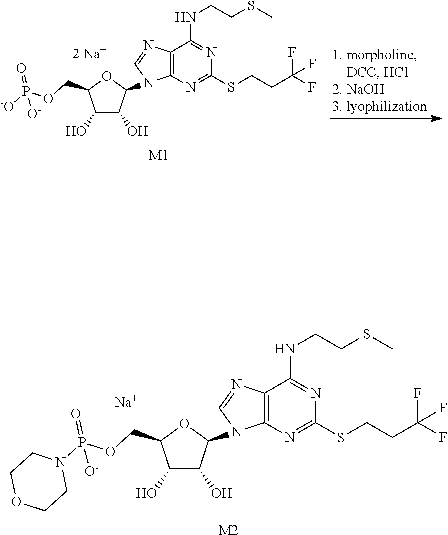

A compound of formula M1 (5.65 g), t-butanol (84.75 mL), and water (84.75 mL) were added to a suitable vessel under nitrogen at 20-30° C. and stirred for 1 hour. An aqueous solution of 1 N HCl (19.04 mL) was slowly added to the reaction mixture at this temperature and stirred for 30 minutes. Morpholine (2.47 mL) was added to the reaction mixture at 20-30° C. and stirred for another 30 min. Then the reaction mixture was heated to 65-70° C. The solution of DCC (5.89 g) in t-butanol (17.0 mL) was slowly added to the mixture at this temperature for 30 minutes. Then the resulting mixture was heated to reflux and stirred for 3 hours. After the reaction was completed, the reaction mixture was cooled to 20-30° C. DMS (7 mL) was added to the reaction mixture first and then the mixture was distillated with PPW swap under reduced pressure till 10 vol. of volume. The slurry solution was cooled to 0-10° C. and filtered after being stirred for 2 hours. An aqueous solution of 1 N NaOH (28.56 mL) was slowly added to the mixture and stirred for 1 hour. MeOH (11.3 mL) and MTBE (226 mL) were added to wash the above basic aqueous solution twice. Then the aqueous solution was heated to 50° C. and stirred for 10 minutes, followed by the addition of IPA (452 mL) at this temperature. The resulting solution was cooled to 20-30° C. and stirred for 1 hour. Finally, the mixture was cooled to 0-10° C. and filtered after being stirred for 1 hour. The resulting filtrate was distilled with PPW swap under reduced pressure till 10 volume of solution remained. The remained solution was lyophilized to provide a compound of formula M2 (4.8 g, 91% yield) as pale skinny to white solid.

### EXAMPLE 3

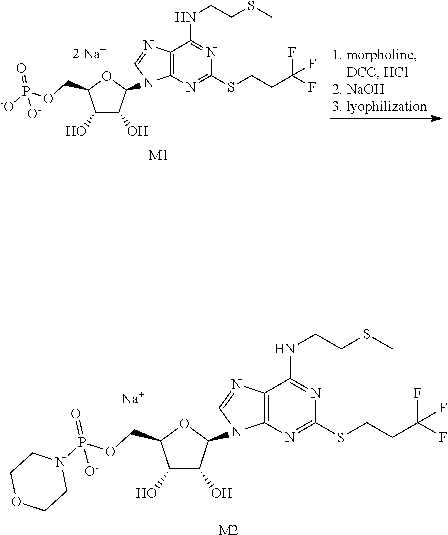

A compound of formula M1 (200 g), t-butanol (3 L), and water (3 L) were added to a suitable vessel under nitrogen at 20-30° C. and stirred for 1 hour. An aqueous solution of 1 N HCl (0.75 kg) was slowly added to the reaction mixture at this temperature and stirred for 30 minutes. Morpholine (87 mL) was added to the reaction mixture at 20-30° C. and stirred for another 30 min. The reaction mixture was heated to 65-70° C. The solution of DCC (208 g) in t-butanol (600 mL) was slowly added to the mixture at this temperature for 30 minutes. Then the resulting mixture was heated to reflux and stirred for 3 hours. After the reaction was completed, the reaction mixture was cooled to 20-30° C. DMS (248 mL) was added to the reaction mixture first and then the mixture was distillated with PPW swap under reduced pressure till 10 vol. of volume. The slurry solution was cooled to 0-10° C. and filtered after being stirred for 2 hours. An aqueous solution of 1 N NaOH (1.04 kg) was slowly added to the mixture and stirred for 1 hour. MeOH (400 mL) and MTBE (8 L) were added to wash the above basic aqueous solution twice. Subsequently, the aqueous solution was heated to 50° C. and then stirred for 10 minutes, followed by the addition of IPA (16 L) at this temperature. The resulting solution was cooled to 20-30° C. and stirred for 1 hour. Finally, the mixture was cooled to 0-10° C. and filtered after being stirred for 1 hour. The resulting filtrate was distilled with PPW swap under reduced pressure till 10 volume of solution remained. The remained solution was lyophilized to provide a compound of formula M2 (213 g, 94% yield) as pale skinny to white solid.

### EXAMPLE 4

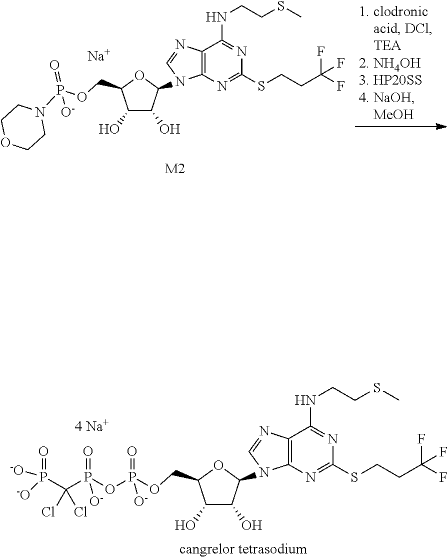

NMP (240 mL) and Et₃N (13.1 mL) were added to a suitable vessel under nitrogen at 20-30° C. Clodronic acid (22.94 g) was slowly added to the mixture, and then stirred for 10 min after being rinsed by NMP (40.0 mL, 1.3 vol.). 4,5-dicyanoimidazole (DCI, 11.06 g) was then added to the above suspended solution at 20-30° C., and stirred for another 10 minutes after being rinsed by NMP (20 mL). The compound of formula M2 (30 g), NMP (280 mL), and DMS (34.4 mL) were added to another 500 mL of three-necked round bottom flask under nitrogen with magnetic stir bar at 20-30° C. and the mixture was stirred at this temperature for 20 minutes. The resulting M2 solution was slowly added to the slurry solution of clodronic acid mixture for 1 hour and rinsed by NMP (20mL). After the reaction was completed, the reaction mixture was slowly charged into the solution of 28% NH₄OH (32.6 mL) in water (2400 mL) at 20-30° C. A crude solution of cangrelor tetraammonium salt was provided in 66% assay yield as pale yellow solution and used for column purification.

The crude solution of cangrelor tetraammonium salt (which was prepared from 30.0 g of the compound of formula M2) was slowly loaded into the HP20SS column. After loading, the column was flushed with 5-85% MeOH in H₂O containing 0.28% NH₄OH and 1% NH₄OAc. Each of the fractions was collected and monitored by HPLC. The fractions containing qualified cangrelor tetraammonium salt were combined and mixed with NaOAc.3H₂O (46.8 g) first. The above mixture was loaded into another HP20SS column and the HP20SS column was flushed with 50% MeCN/H₂O. Each of the fractions was collected and monitored by TLC. The fractions containing cangrelor teraammonium salt (20.49 g of assay weight) were combined and stored in 4° C. for next isolation step.

The fractions containing 20.49 g of assay weight of cangrelor tetraammonium salt and 3 N NaOH_{(aq)} (44 mL) were added into a suitable vessel under nitrogen. The mixture was stirred at 20-30° C. for 10 min. MeOH (425 mL) was slowly added to the above mixture at this temperature for 1 hour. The resulting mixture was then cooled to 0-5° C. Afterwards, the mixture was filtered by vacuum suction and the filtered cake was washed with pre-cooled (0-5° C.) MeOH (200 mL) for three times. The wet cake was purged with nitrogen for 1 hour and dried at room temperature under vacuum to afford cangrelor tetrasodium. (21 g, 53% of yield from the compound of formula M2).

The invention is not limited by the embodiments described above which are presented as examples only but can be modified in various ways within the scope of protection defined by the appended patent claims.

We claim:

1. A process for preparing cangrelor tetrasodium comprising:
a) reacting a compound of formula M1 with morpholine to form a compound of formula M2; and 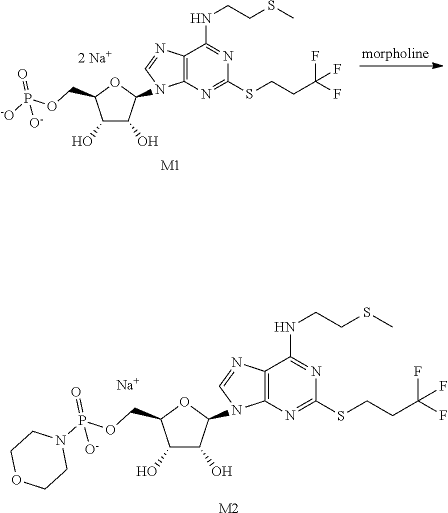
b) reacting the compound of formula M2 with clodronic acid to provide cangrelor tetrasodium

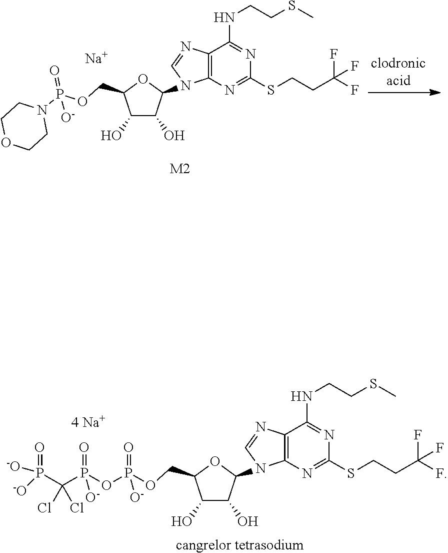

2. The process of claim 1 further comprising a step of phosphorylating a compound of formula SM1 to form the compound of formula M1:

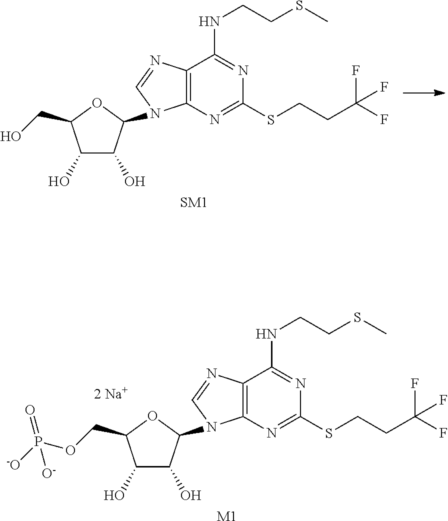

3. The process of claim 2 wherein the compound of formula M1 is isolated by precipitation in a solvent selected from the group consisting of DCM, acetone, THF, MTBE, IPE, EtOAc, IPAc, MeCN, MeOH, EtOH, IPA, t-BuOH, toluene, heptane, cyclohexane, water, and combinations thereof.

4. The process of claim 3 wherein the solvent is one of the following combinations:
1) MeOH and DCM;
2) MeOH and MTBE;
3) MeOH and IPE;
4) MeCN and water;
5) MeCN, water, and acetone; and
6) MeCN, water, and t-BuOH.

5. The process of claim 4 wherein the solvent is the combination of MeCN and water.

6. The process of claim 1 wherein the compound of formula M2 in step a) is modified by a workup technique selected from a group consisting of:
a) DCU filtration,
b) salt-exchanging with NaOH,
c) extraction with MeOH/MTBE or MTBE,
d) phosphate salt elimination, and
combinations thereof.

7. The process of claim 1 further comprising purifying the cangrelor tetrasodium obtained in step b) by a polystyrene/divinylbenzene resin.

8. The process of claim 7 wherein the polystyrene/divinylbenzene resin is selected from the group consisting of HP20, HP20SS, SP20SS, HP21, SP825, SP850, SP70, and combinations thereof.

9. The process of claim 8 wherein the polystyrene/divinylbenzene resin is HP20SS.

10. The process of claim 7, wherein the polystyrene/divinylbenzene resin is eluted by a solution selected from the group consisting of H₂O, MeOH, EtOH, IPA, acetone, MeCN, MeOH/H₂O, EtOH/H₂O, MeCN/H₂O, 0-1% NH₄OH, 0-5% NH₄OAc, 0-1% NaCl, 0-1% NaOAc, 0-3% DMS, and combinations thereof.

11. The process of claim 10, wherein the eluent is MeOH together with water or MeCN together with water.

12. The process of claim 1, wherein the cangrelor tetrasodium formed in in step b) is isolated by precipitation in a solvent selected from the group consisting of EtOH, IPA, DMSO, acetone, MeOH, MeCN, n-PrOH, t-BuOH, MTBE, and combinations thereof.

13. The process of claim 12 wherein the solvent is selected from the following combinations:
1) MeCN/MeOH;
2) MeCN/EtOH;
3) MeCN/IPA;
4) acetone/MeOH;
5) acetone/EtOH;
6) acetone/DMSO;
7) IPA/MeOH;
8) n-PrOH/MeOH;
9) t-BuOH/MeOH; and
10) MeOH/MTBE.

14. The process of claim 12, wherein the solvent is MeCN or MeOH.

15. The process of claim 1 wherein the reacting step b) comprises: b1) reacting the compound of formula M2 with clodronic acid; b2) quenching the reaction mixture obtained in step b1) with ammonium hydroxide (NH₄OH) to obtain cangrelor tetraammonium; and b3) reacting the cangrelor tetraammonium with NaOH to provide cangrelor tetrasodium by salt-exchange:

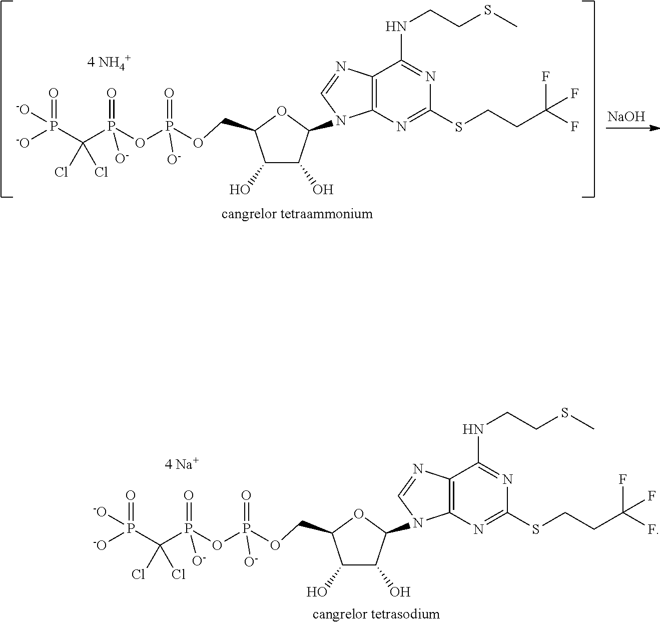

16. The process of claim 6, wherein the workup technique is the phosphate salt elimination conducted in acetone, MeCN, IPA, IPA together with MTBE, or IPA together with water.

17. The process of claim 16, wherein the phosphate salt elimination is conducted in IPA together with water.

18. A compound of formula M2: 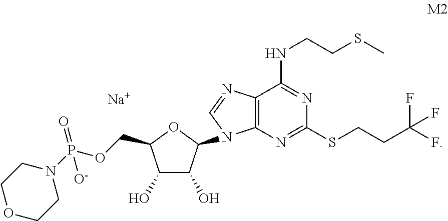 [SMILES: CSCCNc1nc(SCCC(F)(F)F)nc2c1ncn2[C@@H]1O[C@H](COP(=O)([O-])N2CCOCC2)[C@@H](O)[C@H]1O ; [Na+]]
